(19) United States
(12) Patent Application Publication
(10) Pub. No.: US 2004/0102716 A1
(43) Pub. Date: May 27, 2004

(54) Means for stripping blood-lines

(76) Inventors: William Leonard Mobbs, Crescent Farrar (AU); Jagmohanbir Singh Dillon, Oxley (AU)

Correspondence Address: Sidley Austin Brown & Wood, 1501 K Street NW, Washington, DC, 20005, US

(21) Appl. No.: 10/415,318

(22) PCT Filed: Oct. 31, 2001
(86) PCT No.: PCT/AU01/01400

(30) Foreign Application Priority Data
Nov. 2, 2000 (AU) PR 1171

(51) Int. Cl. A61B 5/00

(52) U.S. Cl. 600/578; 604/403

(57) ABSTRACT

A powered hand-held device (200) for use in stripping blood-lines is disclosed comprising a body (202) housing a battery (206) in a handle portion (204) thereof and housing a first motor (210) in a head portion (208) thereof. A pair of rollers extends from the head at an angle to the handle and is drivingly connected to the motor (210) by a drive mechanism (212). One of the rollers (220) is an idler and is mounted on a support member (222) adapted for lateral movement in the head (208) so that it can be moved toward and away from the other roller (not shown). Support member (222) is driven by an actuator including a second motor (214) that rotates a gear (218) which engages a toothed rack (226) on support member (222).

### TECHNICAL FIELD

This invention relates to devices and methods for use in stripping blood-lines that are connected to blood packs.

### BACKGROUND

Accepted procedures for collection of blood from donors involve inserting a needle into the donor's vein and delivering blood into a blood collection bag via a tube that is commonly called the ‘blood-line’. The bag normally contains an anticoagulant and/or a blood preservative solution. After the desired amount of blood has been collected (usually a ‘unit’ of 500 ml), the blood-line is sealed near the needle and the line is cut on the needle side of the seal so that the needle can be safely discarded. Normally the needle will be contained within a guard so as to avoid the danger of needle-stick injury. The blood-line can be sealed by knotting, by heat-sealing or by crimping. Crimping the blood-line to seal it from the needle is normally effected by bending the line double, placing a metal (or plastic) ring or clip over the doubled line and using a device like a pair pliers to flatten the ring or clip onto the tube.

Once the blood-line has been sealed and the needle safely removed, the blood contained in the blood-line is discharged into the bag by pinching the blood-line just above its crimped end, moving the pinch point along the line toward the bag and relieving the pinch on the blood-line to allow it to refill with blood. If desired, the bag can be moved about or squeezed before the pinch is relieved to help mix the blood in the bag. These actions are called ‘stripping’ and may be repeated a few times to ensure that the blood that finally refills the tube contains anticoagulant and/or preservative and is homologous with the blood in the collection bag. After stripping, the blood-line is heat-sealed at intervals to form a string of narrow elongate pouches containing blood. The blood-line (pouched in this way) is retained with the blood bag so that the pouches can be used later for sampling and cross-matching purposes.

Stripping is normally effected by using another pliers-like device that is equipped with a pair of rollers. The device is opened to move the rollers apart, the blood-line is placed between the rollers at a point on the line near the sealed end, the device is closed to pinch the line and the device is pushed upstream (toward the collection bag) while holding the sealed end of the line. As the device moves up the line, it drives blood contained therein into the bag. When the bag is reached, the device is opened to release the line and moved to the sealed end again, allowing the line to once again fill with blood. The process may then be repeated.

Since a phlebotomist in a busy collection center can collect blood from 20-30 donors in one day the burden of stripping the blood-lines is considerable and can easily result in repetition strain injury [RSI] or carpel tunnel syndrome.

### OUTLINE OF THE INVENTION

From one aspect, the present invention comprises a powered hand-held device for use in stripping blood from blood-lines during blood collection procedures, the device having a pair of rollers for pinching a blood-line and which can be driven to move the device along the blood-line. The device includes drive means for one or both rollers and, preferably, actuator means for moving the rollers together and apart. The actuator means can be hand-operated using a lever or the like attached to the body of the device, or it can be powered from within the device under operator control. Preferably, the actuator means is also capable of crimping a ring or staple onto the folded-over end of a blood-line to effect the sealing thereof.

Conveniently, the axes of the rollers are parallel and juxtaposed and arranged at an angle to the handle of device and, also conveniently, one or both rollers are waisted so that the center is of a smaller diameter than the ends to assist in keeping the blood-line between the rollers when the device is in operation. Angles of between 20 and 60 degrees between the roller axes and the handle are envisaged, but an angle of between 40 and 50 degrees has been found to be most convenient. It is also desirable that the roller drive means be reversible so that the device can be driven in either direction along the blood-line being stripped.

Preferably, the pair of rollers comprises a first roller connected to the drive means and fixedly located within the head of the device and a second roller that is an idler and is capable of movement toward and away from the first roller. For that purpose the second roller may be mounted on a support member, which is laterally movable to and fro in the head of the device by the actuator. The support member may conveniently be used to effect the crimping of a ring or staple onto the blood-line. In one arrangement, the same motor that drives the rollers in rotation can be used to effect the lateral movement of the rollers and/or the opening and closing of the crimping jaws, whether these actions are effected separately or jointly. This may be done by including clutch means within the actuator means to operably and selectively connect the actuator means to the motor drive.

Though it will be usual to employ one pair of rollers, one being driven and one idling, more than two rollers may be used and more than one may be driven. For example, two driven rollers and/or a set of three rollers can assist in moving the device along a blood-line with minimal slippage or misalignment.

### DESCRIPTION OF EXAMPLES

Having broadly portrayed the nature of the present invention, a number of examples will now be described with reference to the accompanying drawings, in which:

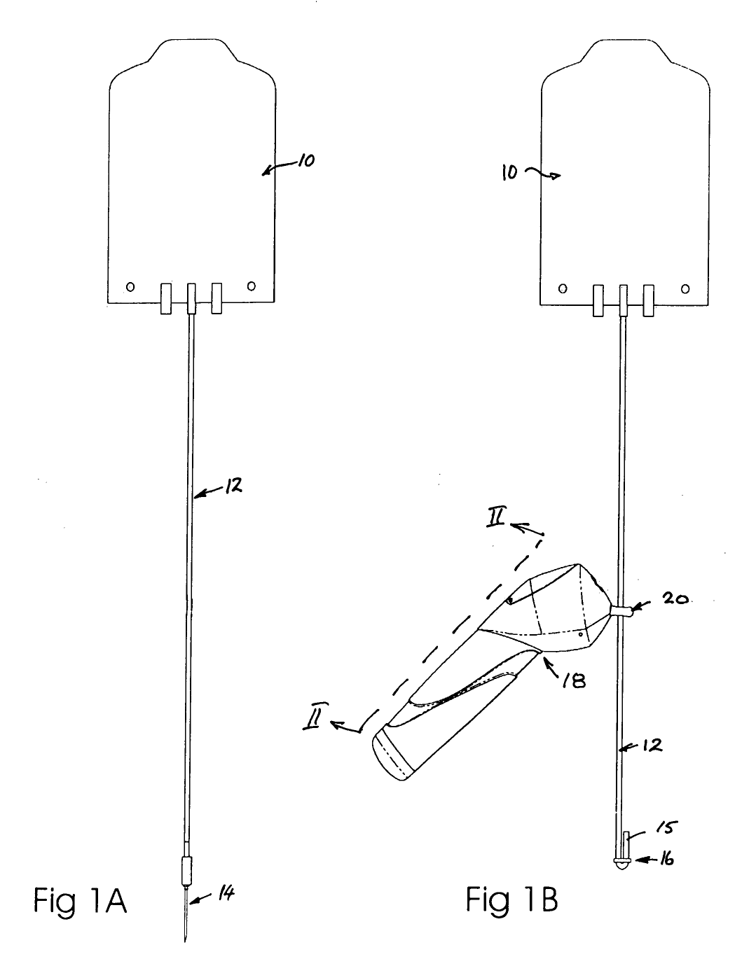

FIG. 1A is a diagrammatic plan view of a blood pack and blood-line with a needle attached, and FIG. 1B is a similar view of the blood pack and blood-line after sealing by crimping and removal of the needle, a stripping device of the first example being shown in use.

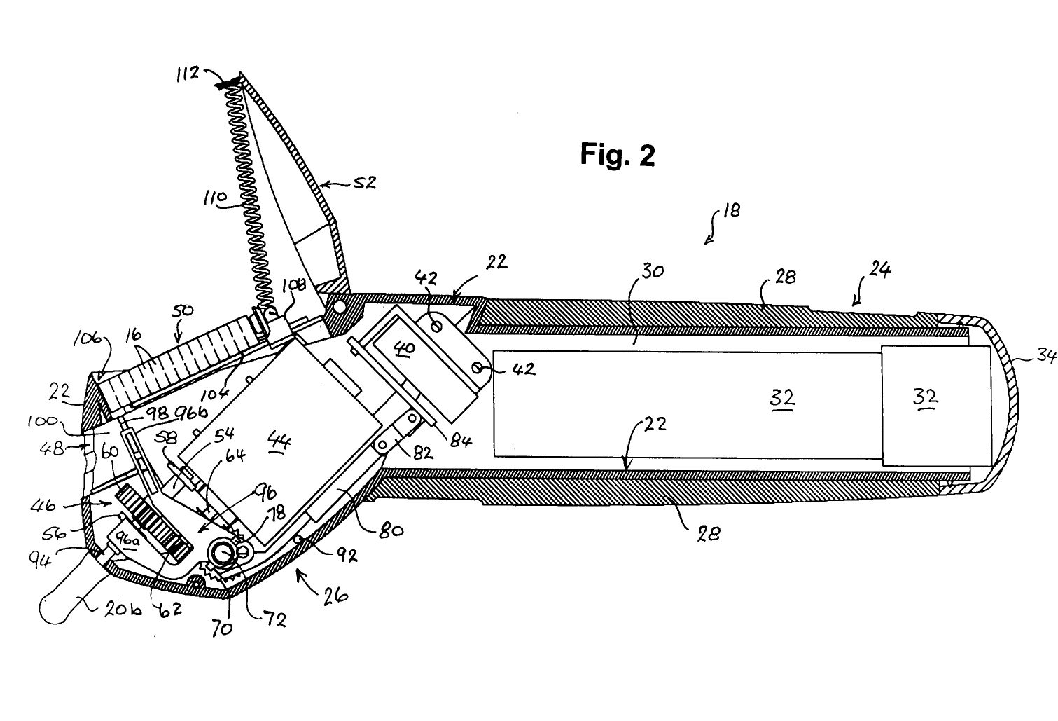

FIG. 2 is a longitudinal section of the device of the first example taken on section II-II indicated in FIG. 1, only the driven roller being shown and internal parts of the device not being shown in section.

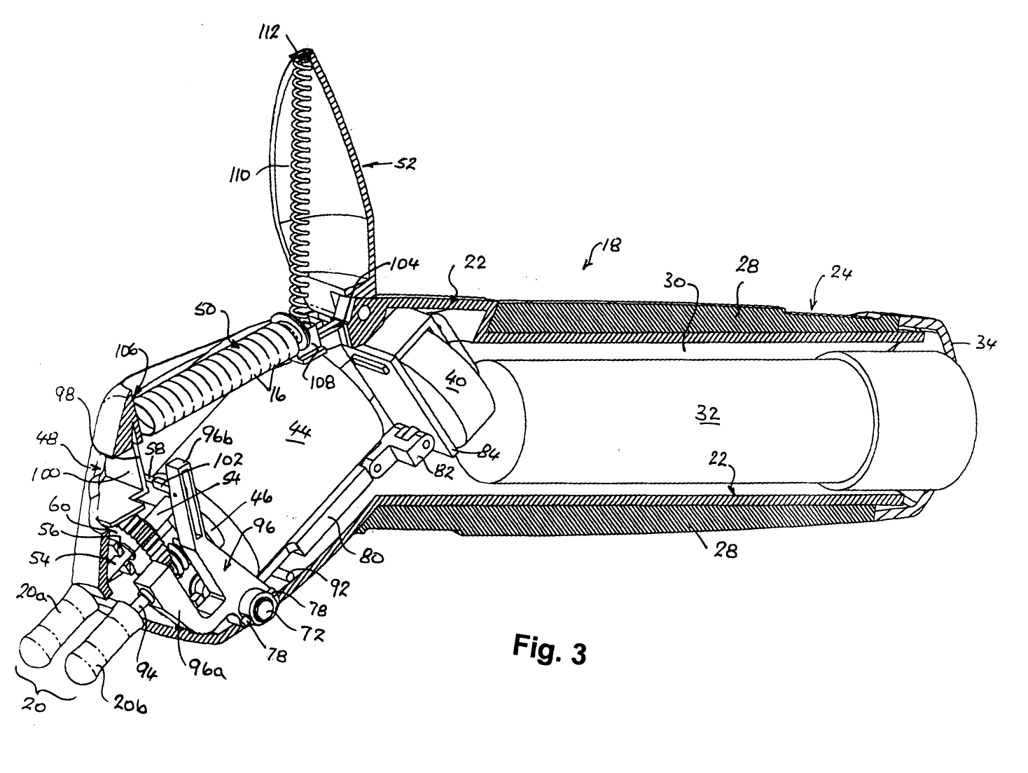

FIG. 3 is a perspective view of the device of the first example taken on section II-II indicated in FIG. 1, both the driven and idler rollers being shown and the body shown sectioned or cut-away to show the internal parts of the device.

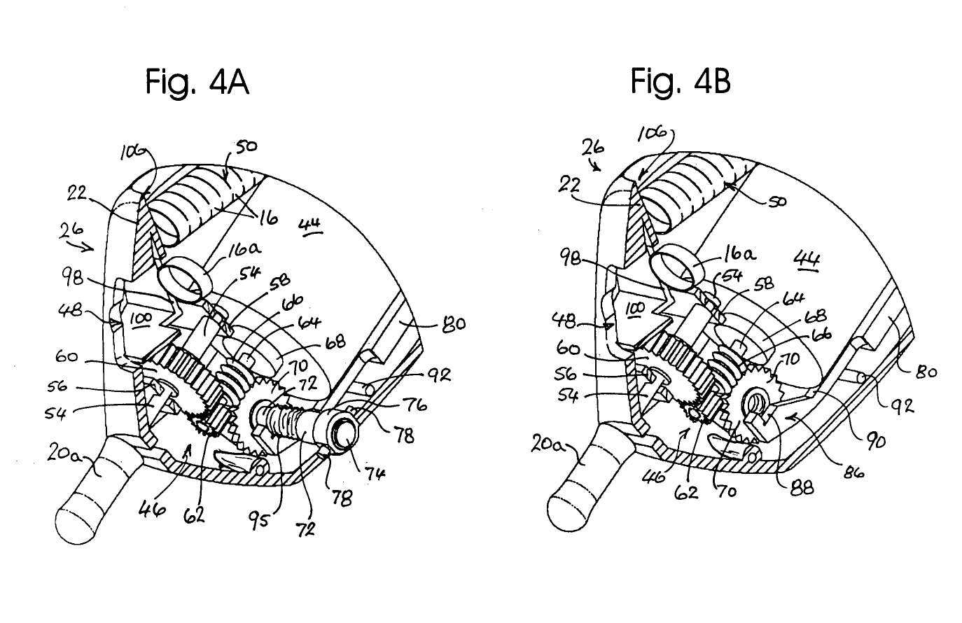

FIGS. 4A and 4B are perspective part-sectional views of the head of the device of the first example with different components removed.

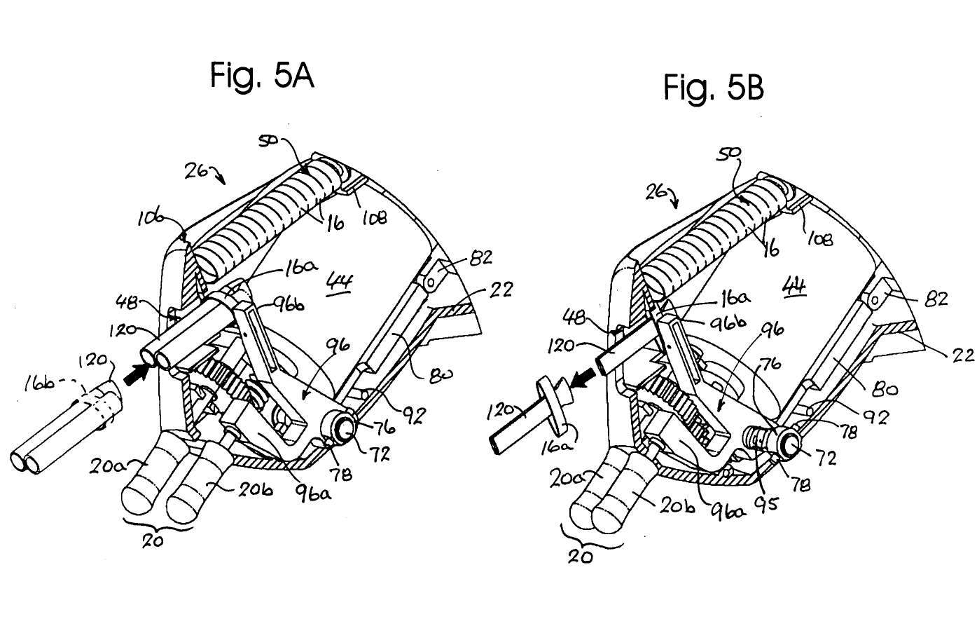

FIGS. 5A and 5B are perspective part-section views of the head of the device of the first example, FIG. 5A showing the folded end of a blood-line being inserted for crimping and FIG. 5B showing the rollers together and the folded blood-line after crimping.

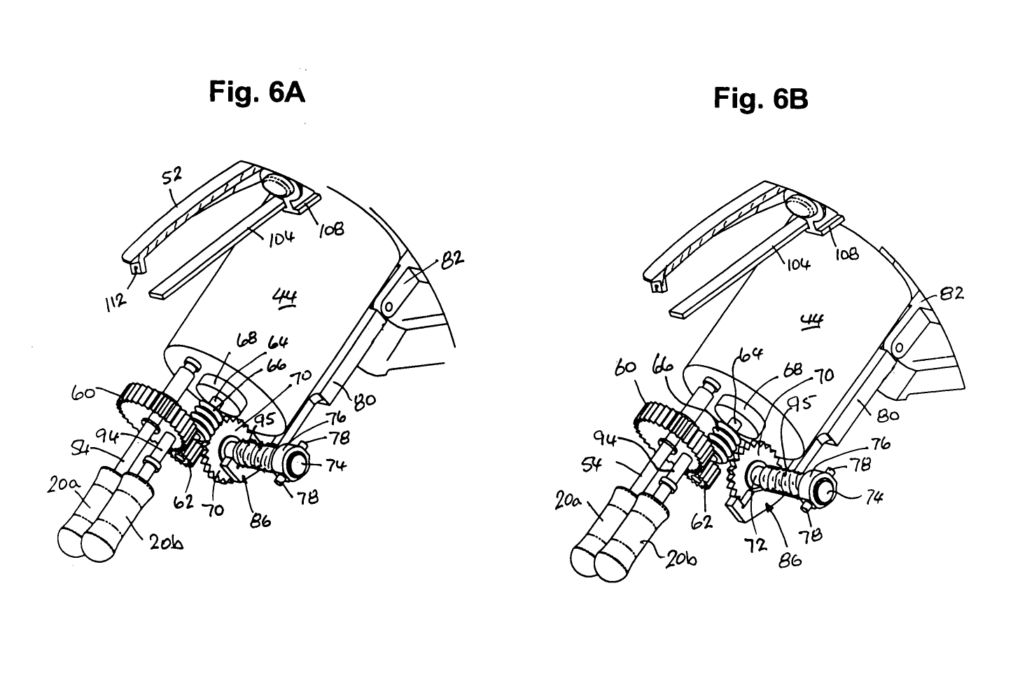

FIGS. 6A and 6B are perspective views of the drive mechanism of the device of the first example showing the mechanism for translating the idler roller engaged and disengaged, respectively.

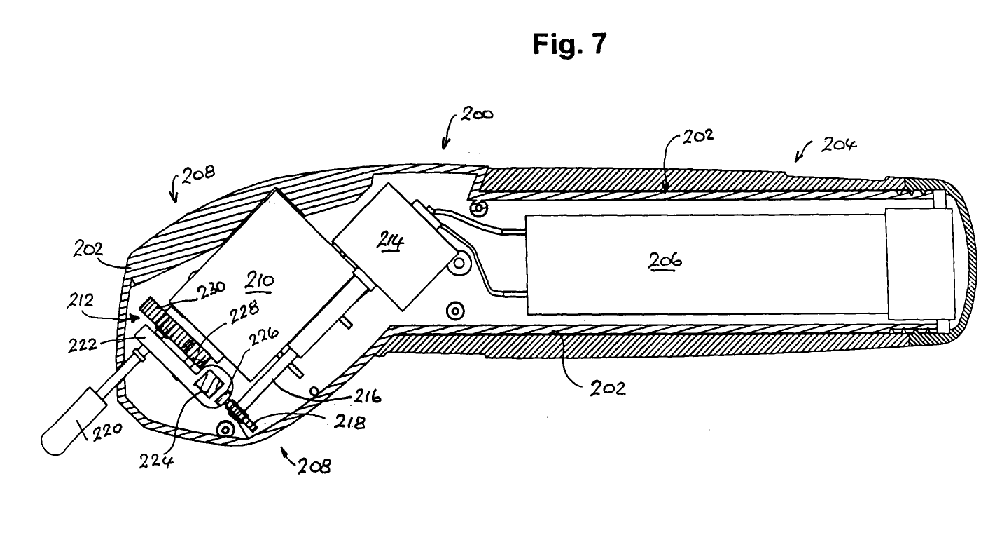

FIG. 7 is a longitudinal sectional elevation of a modified version of the stripping device of the first example, which comprises the second example of this invention.

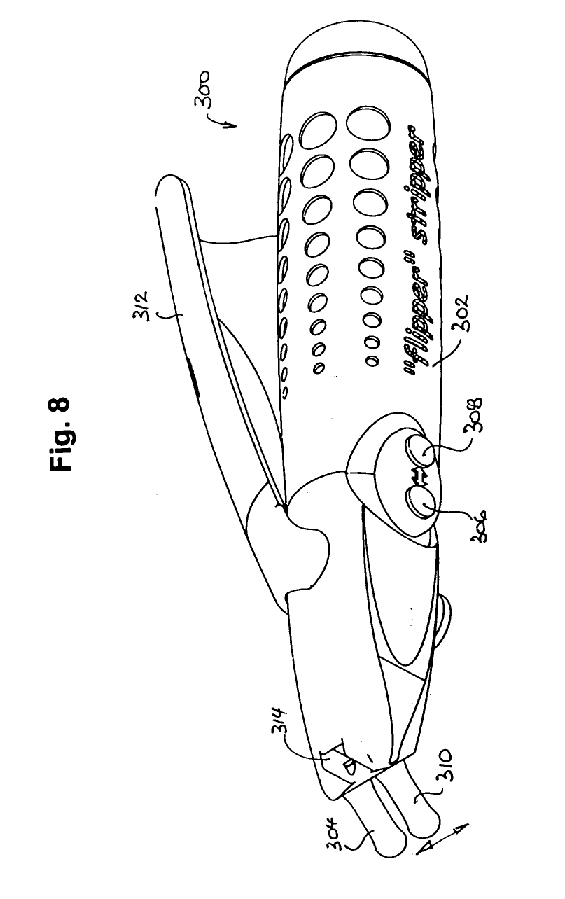

FIG. 8 is an external perspective of the device comprising the second example of the implementation of the invention.

FIG. 1A shows a blood-pack bag 10 with its attached blood-line 12 and needle 14 immediately after needle 14 has been withdrawn from a donor's arm (not shown). To prevent blood flowing out of needle 14, bag 10 is normally arranged below the level of the donor's arm and the needle. Also, either before the needle is withdrawn or immediately thereafter, blood-line 12 is doubled over near needle 14—as shown at 15 in FIG. 1B—to prevent blood from exuding from the needle. The doubled-over portion 15 of line 12 is then sealed by crimping and needle 14 is cut off. It is normal practice for a needle guard (not shown) to be slipped over the needle 14 as soon as it is removed from the donor's arm. The guard protects the phlebotomist against needle-stick injury and catches any drips of blood that may exude from the needle.

FIG. 1B shows the blood-line after it has been doubled over and crimped and after the needle has been cut off and disposed of safely. In this case crimping is effected by passing a metal ring 16 over the doubled tube 15 of the blood-line 12 and flattening it onto the doubled tube 15. Once the tube 12 has been sealed, the phlebotomist (not shown) strips the blood-line by using a stripper device 18 formed in accordance with the present invention. Device 18 has two juxtaposed rollers 20 of waisted shape that are clamped about blood-line 12 and that can be used to drive the device toward bag 10, forcing blood in line 12 into bag 10. When stripper 18 gets near bag 10, the phlebotomist operates the device either to (i) reverse the direction of rollers 20 so that the device is then driven toward the crimped end of blood-line 12 or (ii) to move rollers 20 apart to release line 12 and to allow the device to be manually moved to the crimped end of the blood-line. Whichever method is used, line 12 fills with blood again and the stripping process is repeated once or twice more to ensure that the blood in line 12 is homologous with the blood in the bag. After satisfactory mixing has been effected, device 18 is removed from line 12 and line 12 is heat-sealed at spaced intervals to form sample pouches (not shown in the drawings).

Referring now to FIGS. 2A to 6B, which comprise various views of the device 18 of FIG. 1 B, the device 18 and its mode of operation will now be described in more detail.

Stripping device 18 basically comprises a tubular hollow molded body 22, comprising a rear portion or handle 24 and a front portion or head 26 arranged at an angle of about 45 degrees to the handle, the handle being over-molded with a soft grip 28. The handle forms a compartment 30 for a battery 32, which is closed at its rear end by a removable end-cap 34 through which the battery can be accessed. A solenoid actuator means 40 is mounted by screws 42 in handle 24 immediately in front of battery 32. When energized, solenoid 40 engages clutch means that permits the rollers 20 to be moved together or apart. The roller drive means comprises a rotary DC motor 44 is mounted in head 26 and connected to rollers 20 by a drive mechanism—generally indicated at 46—that will be described in more detail below. Above rollers 20 at the front of head 26, an aperture 48 is formed in body 22 to permit the insertion of the folded end of a blood-line for crimping by means which will also be described in more detail below. Above motor 44 in head 26, a tube 50 of loosely connected crimp-rings or circular staples 16 is housed in a compartment or magazine having a hinged lid 52. The manner in which individual staples 16 are dispensed and crimped will be described below.

The pair of rollers 20 comprise a fixed roller 20a (sometimes hereafter called the second roller) secured to a shaft 54 that is rotatably and axially located by a front bearing plate 56 and a rear bearing plate 58 [see also FIGS. 4A and 4B], shaft 54 carrying a gear wheel 60 fixed thereon that forms part of drive mechanism 46 [FIGS. 3 and 4]. Mechanism 46 also includes a pinion gear 62 mounted on the front end of the shaft 64 of motor 44, a worm gear 66 being fixed to motor shaft 64 between pinion 62 and the motor front bearing 68. Worm 66 can be engaged by a worm wheel 70 that is fixedly mounted on a transverse shaft 72, which is mounted at one end 74 in a bearing 76 that is pivotally mounted in body molding 22 by trunnions 78 so that shaft 72 is orthogonal to motor shaft 64 and can be pivoted toward and away from the motor shaft so as to bring worm wheel 70 into or out of engagement with worm 66.

The pivoting of shaft 72 is controlled by solenoid 40 via a longitudinally extending push-rod 80, the rear of which is attached by a link 82 to the solenoid armature 84 and the front of which is shaped into a wedge-like hook 86 [FIG. 4B]. Hook 86 has an upper land 88 adapted to support pivoting shaft 72 when solenoid 40 is energized and push-rod 80 is pulled rearward, and a lower land 90 adapted to support shaft 72 when solenoid 40 is de-energized and push-rod 80 is moved forward. Preferably, push-rod 80 is spring-biased in the forward direction, a stop-pin 92 being mounted in body 22 to limit the degree of forward motion; the degree of rearward motion being limited by contact between the armature 84 and the core (not shown) of the solenoid. FIGS. 6A and 6B show push-rod 80 in its rearward and forward positions in which gear wheel 70 is engaged and disengaged from worm 66, respectively.

As will be best seen from FIGS. 3 and 5, the other roller 20b, which is capable of lateral translation, is an idler roller and is mounted by its shaft 94 on a bifurcated support member 96 that is, in turn mounted on pivoting shaft 72. [The roller which is capable of translatory movement is sometimes hereafter referred to as the first roller). In fact, member 96 is transversely bored and internally screw-threaded to mate with an externally threaded portion 95 of shaft 72 located between bearing 76 and worm wheel 66 [see FIGS. 4A and 6]. Thus the threaded portion of shaft 72 functions as a leadscrew that translates member 96 (which functions a nut for the leadscrew) and roller 20b back and forth across the head of the device, depending upon the direction of rotation of motor shaft 64 (and, of course, upon whether or not the solenoid 40 is actuated to engage worm wheel 60 and worm 66). Being an idler, roller 20b is free to rotate on its shaft 94, which is fixed into front prong 96a of forked member 96.

The rear fork 96b of member 96 extends upwardly to form a moving jaw or hammer for crushing a crimp ring 16 onto the folded end of a blood tube that has been inserted into aperature 48 in the front face of head 26, the cooperating fixed jaw or anvil being the edge 98 of a metal plate 100 fitted into body molding 22 of head 26. The hammer 96b and idler roller 20b, of course, move together since both are part of or attached to member 96, which travels along leadscrew 95. Hammer 96b in this example is shown slotted to take a horizontal peg 102 that provides a floor or rest upon which a ring 16 can rest, peg 102 being offset from anvil 100 so that it slides closely thereby as hammer 96a is moved to crush the ring.

The tube 50 of rings 16 is supported on a rail 104 [FIG. 3] and is biased against the inner face 106 of head 26 by a slider 108 fitted to rail 104, slider 108 being in biased forward, in turn, by a tension spring 110 stretched to the front tip of lid 52. The action is similar to that of a common stapler for use with stationary. A tooth-like projection 112 is formed on the underside of lid 52 at the front extremity to anchor the front end of spring 110. When lid 52 is closed, tooth 112 rests on the front crimp ring 16. However, by pressing firmly on the lid with a thumb, the user can detach the front ring and load it into position between hammer 96b and anvil 100 [FIGS. 4A and 4B].

It will be appreciated by those skilled in the art that electrical switches for controlling the motor (forwards and reverse) and for controlling the solenoid (off and on) are required. In addition, off/on limit switches may be required for stopping the motor when roller 20b has been moved fully away from, and fully toward, roller 20a. Optionally, separate limit switches or controls may be use for detecting the presence of a ring 16 ready for crimping and for limiting the movement of the hammer 96b when crushing a ring 16. For the sake of clarity, these electrical switches and controls, together with their associated wiring, have been omitted from the drawings. A convenient arrangement for the user is to provide a single push-button for effecting the reversal of the motor (and driven roller) upon each press and a single push-button for opening/closing the rollers upon each successive press. The default or ‘off’ condition of such a device would then be where rollers 20 are fully open or separated and the motor and solenoid are de-energized. The operation of the device of the first example fitted with such control switches will now be described.

After a unit of blood has been collected and the needle has been withdrawn and guarded, the phlebotomist manually strips the blood-line 12 close to the needle 14 and folds that portion of the line, holding it in one hand. She takes up stripper 18 (in the off or default condition) in the other hand and presses lid 52 to dispense one ring or staple 16a [FIGS. 4A, 4B and 5A] from the front end of tube 50 into position between hammer 96b and anvil 100. Then she puts the folded portion of the line (indicated at 120 in FIG. 5A) into aperture 48 in the head 26 of stripper 18 so that it enters the dispensed ring 16a. The phlebotomist then presses the ‘open/close’ push button to energize both solenoid 40 and motor 44 (in the forward direction). As a result of energizing solenoid 40, push-rod 80 is pulled rearward, pivoting shaft 72 so that worm wheel 70 is moved into engagement with worm 66, which is being rotated in the forward direction by motor shaft 64 as a result of energizing motor 44. Shaft 72 is thus rotated to drive support member 96 and hammer 96b to close the rollers 20 and to crimp ring 16a on the folded tube 120. When the limit switch (not shown) that detects full closing of member 96 is actuated, solenoid 40 is de-energized to disengage worm wheel 70 from worm 66, but roller 20a continues to be driven in the forward direction by motor 44.

The phlebotomist then presses the open/close button again to reverse motor 44 and re-energize solenoid 40. This results in the re-engagement of the worm and wheel (66 and 70), the opening of the rollers 20 and the withdrawal of hammer 96a from anvil 100, enabling the crimped folded blood-line 120 to be withdrawn from aperture 48 in the head 26 of device 18. When member 96 reaches the fully open position another limit switch (not shown) is operated to turn off both solenoid 40 and motor 44, returning device 18 to the off or default condition.

With the crimped folded end 120 of tube 12 free of device 18, the phlebotomist places the portion of blood-line 12 that is close to the crimped end between separated rollers 20 of device 18 and presses the open/close button (not shown) to re-close the rollers and drive roller 20a in the forward direction. This clamps blood-line 12 between rollers 20a and 20b and draws blood-line 12 through the roller nip. When the blood bag is reached, the phlebotomist has the choice of pressing the forward/reverse button (not shown) or the open/close button (also not shown in the drawings). If the forward/reverse button is operated, the direction of motor 44 and rollers 20 is reversed to pull the stripped blood-line 12 in the other direction through the roller nip. When, the crimped end of the blood-line is reached, the phlebotomist again has the choice of pressing the forward/reverse or the open/close button; that is, of continuing with the stripping operation or opening the rollers and returning the device to the default condition.

If the open/close button is pressed when the blood bag is reached at the end of the first stripping action, motor 44 is reversed and solenoid 40 is energized so that worm and worm wheel (66 and 70) are engaged to effect the opening of the rollers until the appropriate limit switch (not shown) is actuated and the device 18 is returned to its off or default condition. The phlebotomist can then move the device 18 to the crimped end of the blood-line 12, place the line between the rollers again and press the open/close button to re-close rollers 20 on line 12 and drive rollers 20 in the forward direction again to repeat the stripping action.

An alternative method of crimping the blood-line is indicated in FIG. 5A. Here, instead of using crimp rings or staples 16 from tube 50, as described above, the phlebotomist takes a ring from a container (not shown) and places it over the folded end 120 of the blood-line 12 by hand. Such a ring is indicated in broken lines at 16b in FIG. 5A. The folded end 120 of blood-line 12 with ring 16b fitted thereon is then placed in hole 48 in head 26 of device 18 (when in the off or default condition) so that ring 16b is positioned between hammer 96a and anvil 100. Ring 16b is then squeezed flat in the same manner as described above for ring 16a.

It will be seen that the device of the first example offers considerable benefits to phlebotomists in busy blood collection agencies who are required to strip many blood-lines in a working day or shift.

The second example of the invention is the stripper 200 illustrated in FIG. 7 and is a modification of device 18 of the first example. Device 200 differs from device 18 in that a solenoid and pivoting shaft are not used to drive the support member on which the moving roller is mounted. Instead, a second rotary motor is employed to drive a rack and pinion mechanism to slide the support member on a fixed cross-shaft.

Referring more particularly to FIG. 7, device 200 of the second example again comprises a body molding 202 defining a handle portion 204 that houses a battery 206 and a head portion 208 that houses the main motor 210 and the associated drive mechanism 212. A rotary motor 214 with an internal reduction gear replaces the solenoid of the first example and its drive shaft 216 carries a gear pinion 218. As before, moving idler roller 220 is carried on a support member 222 that is capable of transverse reciprocation to (i) move roller 220 toward and away from a fixed driven roller (not shown in FIG. 7) and to (ii) effect the crimping of a staple or ring onto the folded end of a blood-line (the means for this being similar to that of the first example, but not shown in FIG. 7). However, member 222 is slidingly carried on a square-section transverse shaft 224 that is fixedly mounted in the body molding of head 208, and a toothed rack 226 is formed along the bottom face of member 222 to engage with pinion 218. Thus, member 222 and moving roller 220 can be moved from side to side by suitable forward and reverse actuation of motor 214.

Drive mechanism 212 can thus be greatly simplified with respect to that of the first example since the main motor 210 is not required to move member 222 as well a rotate the driven roller. A pinion 228 is mounted on the motor shaft (not shown) and meshes with a gearwheel 230 mounted on the shaft of the driven roller (not shown). Since no worm or worm wheel is employed (as in the first example) the support member 222 does not need to be bifurcated and drive mechanism 212 can be made more compact.

The third example of the invention is illustrated in external perspective by FIG. 8. It comprises a stripper device 300 comprising a hollow tubular body 302 housing a battery, motor and drive mechanism (not shown) for rotating a driven roller 304 in forward or reverse direction according to the operation of push-buttons 306 and 308. While an idler roller 310 that can be moved transversely to and from driven roller 304 is employed as in the previous examples, its transverse movement is not motor driven. Instead, in this example, the transverse movement of idler roller 310 is effected manually by a lever 312 that is pivoted to body 302 and aligned with the body when both the body and the lever are gripped in one hand, lever 312 being biased toward the extended position shown in FIG. 8. The connection (not shown) between lever 312 and the moving support member (not shown) on which idler roller 310 is mounted is such that, when lever 312 is extended (as illustrated), idler roller 310 is at its furtherest position from driven roller 304, and when lever 312 is aligned with body 302, idler roller 310 is at its closest position to driven roller 304. As in the previous examples, the support member (not shown) on which idler roller 310 is mounted can be used to crimp a staple or ring (not shown in FIG. 8) onto the folded end of a blood-line (also not shown in FIG. 8). This is done by placing the ring on the folded tube (as described with respect to ring 16b of FIG. 5A), holding device 300 with lever 312 extended (as illustrated in FIG. 8), placing the ring and tube in a recess 314 formed in the head of body 302, and gripping the lever 312 to bring it against body 302 and to press the support member on which roller 310 is mounted into recess 314 against the ring to effect its compression or crimping. The crimped ring is released from recess 314 by allowing lever 312 to return to the position illustrated.

While the chosen examples meet the objectives of the invention, it will be appreciated that many variations to these examples can be made, and many other examples are possible, without departing from the scope of the invention as defined by the following claims. For instance, our Australian provisional patent application No. PR1171 upon which this application is based discloses a number of other examples. The disclosure of that application is therefore incorporated within this specification.

1 A powered hand-held device for use in stripping blood from blood-lines during blood collection procedures, the device having a first roller rotatable about a first axis and a second roller rotatable about a second axis, roller drive means for rotating at least one of the rollers about its axis, and roller actuator means for moving at least the first roller toward and away from the second roller, whereby the first roller can be moved away from the second roller by said actuator means to permit a blood-line to be stripped to be placed between the rollers and whereby said first roller can be moved toward the second roller by said actuator means to compress and grip a blood-line placed there-between, and whereby rotation of at least one of the rollers by said drive means results in the device being driven along a blood-line placed and gripped therebetween.

2 A device according to claim 1 wherein said drive means is adapted to rotate at least one of said rollers in both a forward and a reverse direction about its axis whereby, rotation of said one roller in the forward direction results in the device being driven in one direction along a blood-line placed and gripped between the rollers and whereby rotation of said one roller in the reverse direction results in the device being driven in the other direction along the blood-line placed and gripped between the rollers.

3 A device according to claim 1 or 2, wherein said rollers are arranged in spaced juxtaposition and wherein said actuator means is adapted to move the first roller toward and away from the second roller while maintaining said first and second axes in substantially parallel disposition as the rollers approach one another.

4 A device according to any preceding claim wherein said second axis and second roller are fixed relative to the device, said first axis and first roller are moveable with respect to the device, and wherein said drive means is connected to rotate the second roller about the second axis.

5 A device according to any preceding claim having actuator means for effecting the movement of said first roller to and from said second roller.

6 A device according to any preceding claim wherein said first roller is mounted on a support member arranged for movement within the device to and fro in a lateral direction that is substantially orthogonal to said second axis, whereby lateral movement of said support means effects the movement of the first roller to or from the second roller.

7 A device according to any one of claims 1-4 wherein said first roller is mounted on a support member arranged for movement within the device to and fro in a lateral direction that is substantially orthogonal to said second axis, whereby lateral movement of said support means effects the movement of the first roller to or from the second roller, and wherein the device includes actuator means for effecting said to and fro movement of said support member.

8 A device according to claim 7 having clutch means operable to selectively connect said drive means to said actuator means so that operation of the drive means effects movement of said support member when said clutch means is operated.

9 A device according to claim 8 wherein said actuator means comprises a laterally disposed leadscrew and nut assembly in which either the leadscrew or the nut is an element joined to said support member for movement therewith, and wherein said clutch means is adapted to selectively connect said assembly with said roller drive means to effect the movement of the support member.

10 A device according to claim 8 or 9 wherein said clutch means is electrically operable by first switch means on the exterior of the device to effect the movement of the support means and the first roller to and from the second roller.

11 A device according to claim 7 wherein said actuator means comprises a first electrical motor connected to drive the support member, and wherein said first motor is electrically operable by first switch means on the exterior of the device to effect the movement of the support means and the first roller toward and away from the second roller.

12 A device according to claim 7 comprising a body housing said drive means, and wherein said actuator means comprises a lever hingedly attached to the exterior of said body and operable by the user of the device to move the support means toward said second roller when gripping the body in one hand.

13 A device according to any one of claims 7 to 12 having crimping means adapted to crimp or compress a ring or staple onto the folded-over end of a blood-line to seal it, said crimping means comprising an aperture within the device adapted to accept a folded-over end of a blood-line with an uncompressed ring or staple fitted thereon, an anvil against which a ring or staple can be crimped, and a hammer or moving jaw forming part of, or connected with, said support member, so that movement of the support member to or from said second roller can be used to effect crimping movement of said hammer or moving jaw.

14 A device according to any one of claims 7 to 12 having:
crimping means adapted to crimp or compress a ring or staple onto the folded-over end of a blood-line to seal it, said crimping means comprising an aperture within the device adapted to accept a folded-over end of a blood-line, an anvil against which a ring or staple can be crimped, and a hammer or moving jaw forming part of, or connected with, said support member, and
means within the device for selectively feeding a ring or staple into said aperture between said anvil and said hammer or moving jaw so that the folded-over end of a blood-line will enter said staple or ring selectively fed into the aperture,
so that movement of the support member to or from said second roller can be used to effect crimping movement of said hammer or moving jaw on the staple or ring and the folded-over blood-line.

15 A device according to any preceding claim wherein the drive means comprises a reversible electrical motor operable by second switch means on the exterior of the device to effect the rotation of said second roller about said second axis in the forward and in the reverse direction.

16 A hand-held device for use in stripping blood from blood-lines during blood collection procedures, the device comprising:
a body having an elongatge tubular handle adapted to house a battery and having a head extending forward from the handle, said handle having a central axis,
first and second rollers mounted on said head to extend therefrom in parallel juxtaposed relationship at an angle to the axis of said tubular handle, actuator means adapted to move said rollers away from one another while maintaining said parallel juxtaposed relationship so as to allow a blood-line to be placed therebetween and adapted to move said rollers toward one another so as to pinch said blood-line therebetween, and
a first electrical motor adapted to be powered by said battery and to drive at least one of said rollers so as to drive the device along a blood-line pinched between the rollers to effect the stripping thereof.

17 A device according to claim 16 wherein said actuator means comprises a lever attached to the body and adapted to be operated when gripping the body with one hand.

18 A device according to claim 16 wherein said actuator means comprises a second electrical motor adapted to be powered by said battery.

19 A device according to any one of claims 16 to 18 incorporating crimping means for crimping a ring or staple on a blood-line to effect the sealing thereof, and characterized in that said crimping means is operated by said actuator means.

20 A device according to claim 19 wherein the crimping means is located within said head and includes (i) a magazine for storing a plurality of rings or staples and (ii) feeding means for feeding single staples or rings into a position where the folded-over end of a blood-line can be inserted therein and where the ring or staple can be crimped onto the blood-line by operation of said actuator means.
